(19) United States
(12) Patent Application Publication
(10) Pub. No.: US 2009/0069253 A1
(43) Pub. Date: Mar. 12, 2009

(54) WHITENING COSMETIC COMPOSITION CONTAINING ARBUTIN NANOPARTICLES

(75) Inventor: Teruyuki NANBU, Hakata-ku (JP)

Correspondence Address: OBLON, SPIVAK, MCCLELLAND MAIER & NEUSTADT, P.C., 1940 DUKE STREET, ALEXANDRIA, VA, 22314, US

(73) Assignee: ASKA CORPORATION Co., Ltd., Hakata-ku (JP)

(21) Appl. No.: 12/052,192

(22) Filed: Mar. 20, 2008

(30) Foreign Application Priority Data
Sep. 11, 2007 (JP) 2007-235022

(51) Int. Cl. A61K 31/7034; A61Q 19/02

(52) U.S. Cl. 514/25

(57) ABSTRACT

The present invention provides a whitening cosmetic composition containing arbutin at a high concentration, whereby an improved whitening effect can be attained. In the arbutin-containing whitening cosmetic composition, arbutin is in the form of nanoparticles obtained through a nanoparticle-forming treatment of an aqueous arbutin dispersion. The composition preferably contains arbutin nanoparticles having a particle size of 10 to 150 nm in an amount of 1 to 15 wt. %.

### BACKGROUND OF THE INVENTION

1. Field of the Invention

The present invention relates to a cosmetic composition, and more particularly to a cosmetic composition containing arbutin, and exhibiting a whitening effect; i.e., an effect of alleviating skin problems such as age spots, dullness, and pigmentation.

2. Background Art

Arbutin (hydroquinone glucoside), which exhibits a melanin production inhibitory effect, has been incorporated into whitening cosmetic products (see, for example, Japanese Patent Application Laid-Open (kokai) Nos. 2004-26657 and 2005-289880).

However, arbutin exhibits low solubility in an aqueous solvent (e.g., water), and thus is difficult to incorporate into a cosmetic product at a high concentration. Therefore, the aforementioned conventional whitening cosmetic products containing arbutin encounter difficulty in attaining satisfactory whitening effect.

Japanese Patent Application Laid-Open (kokai) No. 2004-359587 discloses a method for producing emulsified nanoparticles, the method including incorporating a specific amount of arbutin into lecithin, followed by a treatment for forming nanoparticles (hereinafter the treatment may be referred to as a “nanoparticle-forming treatment”), so that the emulsified nanoparticles contain a bioactive substance such as a whitening material in each particle. However, this patent document does not describe the relation between solubility of arbutin and whitening effect.

### SUMMARY OF THE INVENTION

In view of the foregoing, an object of the present invention is to provide a whitening cosmetic composition containing arbutin at a high concentration, whereby an improved whitening effect can be attained.

The present inventors have conducted extensive studies on a whitening cosmetic composition containing arbutin at a high concentration, and as a result have found that when an aqueous arbutin (powder) dispersion is subjected to a nanoparticle-forming treatment, and then incorporated into a cosmetic composition, the arbutin concentration of the cosmetic composition can be increased. The present invention has been accomplished on the basis of this finding.

Accordingly, the present invention provides a whitening cosmetic composition containing arbutin, wherein arbutin is in the form of nanoparticles obtained through a nanoparticle-forming treatment of an aqueous arbutin dispersion.

According to the present invention, since arbutin can be incorporated into a whitening cosmetic composition at a high concentration, whitening effect of the cosmetic composition, which is attributed to arbutin, is further improved.

### DETAILED DESCRIPTION OF PREFERRED EMBODIMENTS

The present invention will next be described in more detail.

No particular limitation is imposed on the type of arbutin. Thus, the arbutin employed in the present invention may be α-arbutin or β-arbutin. Also, the present invention may employ a mixture of α-arbutin and β-arbutin.

In the present invention, arbutin (powder) is dispersed in water, and the resultant aqueous dispersion is subjected to a nanoparticle-forming treatment. The nanoparticle-forming treatment may be carried out by means of a commercially available Nanomizer (e.g., Nanomaker, product of Nanomax).

Through such a nanoparticle-forming treatment, arbutin powder can be transformed into nanoparticles having a size (diameter) of 10 to 150 nm. The resultant aqueous dispersion is transparent, and may contain arbutin nanoparticles in an amount of 1 to 15 wt. %. Even when the aqueous dispersion is stored at low temperature, arbutin is not precipitated. The whitening cosmetic composition of the present invention preferably contains arbutin nanoparticles in an amount of 3 to 15 wt. %.

The whitening cosmetic composition of the present invention which contains, as an essential component, arbutin nanoparticles formed through the aforementioned treatment may also contain other ingredients as additives. Examples of such additives include vitamin C and Mori Cortex extract.

### Examples

The present invention will next be described by way of examples, which should not be construed as limiting the invention thereto.

Cosmetic compositions were prepared using the components shown in Table 1 in amounts also indicated therein. In Comparative Examples 1 to 3, β-arbutin powder was thoroughly dispersed in water. In Examples 1 and 2, β-arbutin powder was dispersed in water in an amount shown in Table 1, and the resultant aqueous arbutin dispersion was subjected to a nanoparticle-forming treatment by means of Nanomaker (product of Nanomax). The thus-formed arbutin nanoparticles were found to have a size of 10 to 150 nm.

In order to evaluate solubility of arbutin, each of the cosmetic compositions prepared in Comparative Examples 1 to 3 and Examples 1 and 2 was stored at 20° C. or 0° C. for 24 hours, and the state of the cosmetic composition after storage was visually observed. The results are shown in Table 1.

Next, in order to evaluate the whitening effect of each of the cosmetic compositions prepared in Comparative Examples 1 to 3 and Examples 1 and 2, the cosmetic composition was used by 20 female monitors once a day for one month. The cosmetic composition was evaluated using the following four criteria:

A: no whitening effect,

B: slight whitening effect,

C: fair whitening effect, and

D: prominent whitening effect.

The results are also shown in Table 1.

TABLE 1

| | | Comparative | Comparative | Comparative | | |
|---|---|---|---|---|---|---|
| | | Example 1 | Example 2 | Example 3 | Example 1 | Example 2 |
| Composition | Arbutin powder | 7 wt. % | 10 wt. % | 15 wt. % | — | — |
| | Arbutin nanoparticles | — | — | — | 10 wt. % | 15 wt. % |
| | Water | Balance | Balance | Balance | Balance | Balance |
| | Total | 100 wt. % | 100 wt. % | 100 wt. % | 100 wt. % | 100 wt. % |
| Solubility | 20° C. | Dissolved to | Partially | Insoluble | Dissolved to | Dissolved to |
| | | transparent | insoluble | | transparent | transparent |
| | 0° C. | Precipitation of | Insoluble | Insoluble | Dissolved to | Dissolved to |
| | | crystals | | | transparent | transparent |
| Whitening | A | 15 | 5 | 2 | 0 | 0 |
| effect | B | 5 | 10 | 15 | 0 | 0 |
| | C | 0 | 5 | 3 | 12 | 10 |
| | D | 0 | 0 | 0 | 8 | 10 |

As is clear from the data shown in Table 1, the whitening cosmetic compositions of the present invention, which contain arbutin nanoparticles, maintained a transparent state in which arbutin is dissolved, although they had a high arbutin concentration, and exhibited excellent whitening effect.

The present invention finds wide utility in the cosmetic industry, and is also useful in the field of chemistry.

What is claimed is:

1. A whitening cosmetic composition containing arbutin, wherein arbutin is in the form of nanoparticles obtained through a nanoparticle-forming treatment of an aqueous arbutin dispersion.

2. A whitening cosmetic composition as described in claim 1, which contains arbutin nanoparticles having a particle size of 10 to 150 nm in an amount of 1 to 15 wt. %.
